(19) United States
(12) Patent Application Publication
(10) Pub. No.: US 2006/0089569 A1
(43) Pub. Date: Apr. 27, 2006

(54) Articulator with adjustable stiffness distal portion

(76) Inventors: Thomas M. Soukup, Plymouth, MN (US); Gregory L. Townsend, Plymouth, MN (US)

Correspondence Address: Allen W. Groenke;Fredrikson & Byron, P.A., Suite 4000, 200 South Sixth Street, Minneapolis, MN, 55402, US

(21) Appl. No.: 10/973,317

(22) Filed: Oct. 26, 2004

(51) Int. Cl. A61M 25/00

(52) U.S. Cl. 600/585

(57) ABSTRACT

Methods and devices for performing minimally invasive surgical procedures are disclosed. An articulator in accordance with one exemplary embodiment comprises a first wire, a second wire, and a third wire. The third wire being disposed within a first lumen defined by the first wire and the second wire being disposed in a third lumen defined the third wire. A distal portion of the second wire is fixed to a distal portion of the second wire and a distal portion of the third wire is fixed to a distal portion of the first wire.

### FIELD OF THE INVENTION

The present invention relates generally to methods and devices for performing surgical procedures. More particularly, the present invention relates generally to methods and devices for performing minimally invasive surgical procedures.

### BACKGROUND OF THE INVENTION

Intravascular catheters are currently utilized in a wide variety of minimally invasive medical procedures. Generally, an intravascular catheter enables a physician to remotely perform a medical procedure by inserting the catheter into the vascular system of the patient at an easily accessible location and navigating the tip of the catheter to a desirable target site. By this method various target sites in the patient's vascular system may be remotely accessed, including the coronary, cerebral, and peripheral vasculature.

Intravascular catheters are often used in conjunction with a guidewire. When this is the case, the guidewire may be advanced through the patient's vasculature until its distal tip has reached a desired target location. In many cases, the guidewires path through the vascular system will be tortuous, requiring the guidewire to change direction many times. By pushing and rotating the proximal end of the guidewire outside of the patient, the physician attempts to direct the distal end of the guidewire to the desired target site. Once the distal portion of the guidewire is proximate the desired location, the catheter may be threaded onto the guidewire and urged distally until the distal end of the catheter is proximate the target location.

Typically, the catheter enters the patient's vasculature at a convenient location such as a blood vessel in the neck or near the groin. Once the distal portion of the catheter has entered the patient's vascular system, the physician may urge the distal tip forward by applying longitudinal forces to the proximal portion of the catheter. In order for the catheter to effectively communicate these longitudinal forces and resist kinking intravascular catheters are typically quite stiff.

Physicians are often concerned with avoiding excessive hemodynamic interactions between intravascular devices and the walls of the human vascular system. The introduction of intravascular devices into the human vasculature may produce slight to severe damage to vessel walls. Further complicating the procedure is the length of time that a patient is subjected to these interventions. Prolonged procedures where multiple devices are introduced and withdrawn from the vasculature system negatively contribute to the traumatic effects on patient's vasculature.

As an intravascular device is advanced within a blood vessel, it may scrape the fragile layer of endothelial cells which naturally coat the walls of the artery. The function of the endothelial cell layer is to ensure the smooth and steady flow of blood thru the vasculature system. Aggravation and or removal of the endothelial layer can result in unprotected areas within the vasculature that then allows fibrin and platelets to become active. Active fibrin and platelets begin entrapping blood elements producing thrombetic reactions.

Stylets are an additional example of an intravascular medical device. The assignee of the present application has itself previously described a steerable stylet that includes a stylet assembly and a handle. See, U.S. Pat. No. 6,776,765. The stylet assembly has a distal end portion and a proximal end portion and includes a stylet wire having a lumen and a core wire positioned within the lumen with the distal end portion secured to the stylet wire proximate the distal end portion of the stylet wire. The handle includes a hand-held housing structure connected to one of the proximal end portion of the stylet wire or the core wire. In one embodiment, an adjustable tensioner is connected to the other of the proximal end portion of the stylet wire or the core wire to adjust a relative tension force applied between the stylet wire and the core wire. A tension limiter is arranged to limit the tension force to a limit force that is less than a breaking stress force of the stylet wire when the stylet wire is positioned within the lumen of the intravascular device.

In its U.S. Pat. No. 6,755,794 the assignee of the present application has also previously described an adjustable stylet that includes a core wire having a portion surrounded by a compression member preferably comprised of a flat wire spring. Depending upon the configuration, compression or relaxation of the compression member in response to forces at the tip or handle of the stylet results in adjustments to the characteristics of the stylet, including its stiffness and/or length.

It would be highly desirable to have a placement device that is adapted with the ability to be both steered and have its stiffness adjusted.

### SUMMARY OF THE INVENTION

The present invention relates generally to methods and devices for performing surgical procedures. More particularly, the present invention relates generally to methods and devices for performing minimally invasive surgical procedures.

In particular, the invention provides an articulating device (“articulator”) adapted to be used within the body in a minimally invasive fashion. Various characteristics of the articulator can be controlled and adjusted, including its course and shape, its stiffness, and/or its length, in order to facilitate its placement, and in turn the placement or positioning of associated components, such as internal or external catheters and/or devices adapted to be positioned or deployed along the length of the articulator in the course of its use.

An articulator in accordance with one exemplary embodiment of the present invention comprises a first wire and a second wire. In some implementations, a portion of the second wire is at least partially disposed within a lumen defined by the second wire. In some useful implementations, the first wire comprises a distal portion, a proximal portion, and an intermediate portion disposed between the distal portion and the proximal portion. In some embodiments, the proximal portion of the first wire comprises a solid portion of a wall of the first wire.

In certain implementations, the distal portion of the first wire may comprise a coil formed by winding a length of wire into a generally helical shape. In certain other implementations, the distal portion of the first wire comprises a wall defining a cut. The cut may have, for example, a generally helical shape. When this is the case, the cut may define a plurality of turns. The turns may be disposed with spaces between adjacent turns.

In some implementations, the distal portion of the articulator is capable of assuming shapes having various lengths. In some useful methods in accordance with the present invention, the dimension of distal portion of articulator can be varied by urging relative motion between the first wire and the second wire. Also in some useful methods in accordance with the present invention, a lateral stiffness of the distal portion of the articulator can be varied by urging relative motion between the first wire and the second wire.

In some implementations, an intermediate portion of the first wire comprises a wall defining a plurality of slots. When this is the case, a rib of the intermediate portion may be defined by each adjacent pair of slots. In some useful implementations, the intermediate portion of the first wire is capable of assuming one or more generally curved shapes.

An articulator in accordance with another exemplary implementation of the present invention comprises a first wire, a second wire and a third wire. A portion of third wire may be disposed within a first lumen defined by the first wire. The second wire may be disposed within a third lumen defined by the third wire. A distal portion of the second wire may be fixed to a distal portion of first wire at a first joint. A distal portion of third wire may be fixed to first wire at a second joint.

In some useful embodiments of the present invention, relative movement of the proximal end of the second wire relative to the proximal end of the first wire causes the distal portion of the first wire to contract in length. Also in some useful embodiments of the present invention, relative movement between the proximal end of the third wire and the proximal end of the first wire causes the intermediate portion of the first wire to assume a generally bent shape.

An articulator of the present invention can be used in a variety of medical procedures, including urethral catheterization procedures, and endovascular procedures in which initial access is gained through percutaneous needle puncture or open exposure. For use in cardiovascular access, for instance, femoral access can be used with a retrograde approach to provide access to the aorta and its branches. Femoral access with an antegrade approach can be used to obtain access to the ipsilateral infrainguinal. Similarly, brachial or axillary puncture site access can be used with a retrograde approach to obtain access to the aorta and its branches. Alternative sites include the left subclavian, retrogeniculate popliteal, common carotid, and translumbar sites, for access to the ipsilateral SFA, the aorta, the carotid bifurcation, and the aorta, respectively.

### DESCRIPTION OF THE DRAWINGS

FIG. 1 is a side view of an articulator comprising a first wire and a second wire.

FIG. 2 is a cross sectional view of articulator shown in the previous figure.

FIG. 3 is a side view of articulator shown in the previous figure.

FIG. 4 is a side view of an articulator comprising a first wire, a second wire and a third wire.

FIG. 5 is a side view of articulator shown in the previous figure.

FIG. 6 is a side view of the articulator shown in the previous figure.

FIG. 7 is a ventral view of a patient.

FIG. 8 is a partial cross sectional view of a heart.

FIG. 9 is an additional partial cross sectional view of a heart shown in the previous figure.

### DETAILED DESCRIPTION

An articulator of the present invention can be advanced into various body lumens to facilitate the performance of various minimally invasive medical procedures. Examples of body lumens include blood vessels, tear ducts, lymph vessels, lumens for the passage of bile, lumens for the passage of urine, and gastrointestinal lumens. Examples of minimally invasive medical procedures include percutaneous transluminal coronary angioplasty (PCTA), endoscopic retrograde cholangio-pancreaticography (ERCP), endovascular treatment of brain aneurysms, atherectomy procedures, biopsy procedures, and stenting procedures.

An articulator of the present invention can be used to position and/or deliver a variety of medical devices. The medical device used in conjunction with the articulator can be positioning in any suitable manner with respect to the articulator, including within, surrounding, along the length of, or axially concentric with the articulator (e.g., within or surrounding the articulator itself), axially adjacent the articulator, or positioned in one more predetermined positions along the length of the articulator (e.g., distally or along its length). Examples of medical devices that may be used in conjunction with an articulator in accordance with the present invention include catheters, leads, stents, biopsy tools and angiographic die injection catheters.

Various characteristics of the articulator can be controlled and adjusted, including its course and shape, its stiffness, and/or its length, in order to facilitate its placement, and in turn the placement or positioning of associated components, such as internal or external catheters and/or devices adapted to be positioned or deployed along the length of the articulator in the course of its use. The following detailed description should be read with reference to the drawings, in which like elements in different drawings are numbered identically. The drawings, which are not necessarily to scale, depict selected embodiments and are not intended to limit the scope of the invention. Examples of constructions, materials, dimensions, and manufacturing processes are provided for selected elements. All other elements employ that which is known to those of skill in the field of the invention. Those skilled in the art will recognize that many of the examples provided have suitable alternatives that can be utilized.

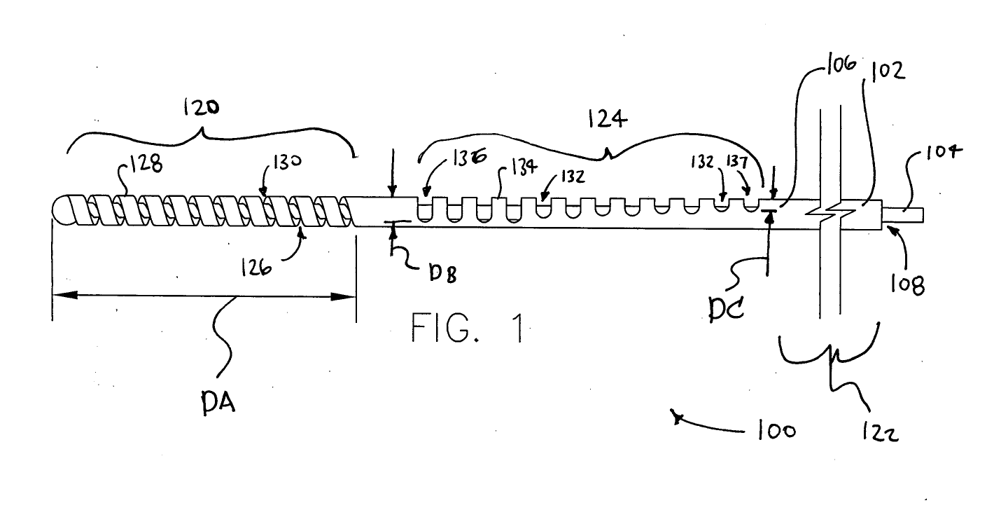

FIG. 1 is a side view of an articulator 100 comprising a first wire 102 and a second wire 104. First wire 102 comprises a wall 106 defining a lumen 108. In the embodiment of FIG. 1, a portion of second wire 104 is disposed within lumen 108 defined by first wire 102. Also, in the embodiment of FIG. 1, first wire 102 of FIG. 1 comprises a distal portion 120, a proximal portion 122, and an intermediate portion 124. In the embodiment of FIG. 1, proximal portion 122 of first wire 102 comprises a solid portion of wall 106.

In the embodiment of FIG. 1, distal portion 120 of first wire 102 comprises a portion of wall 106 that defines a cut 126. In FIG. 1, cut 126 is shown having a generally helical shape. With reference to FIG. 1, it will be appreciated that cut 126 defines a plurality of turns 128. In the embodiment of FIG. 1, turns 128 are disposed with spaces 130 disposed between adjacent turns 128. A dimension DA of distal portion 120 of articulator 100 is illustrated using dimension lines in FIG. 1.

In some embodiments of the present invention the distal portion 120 of articulator 100 is capable of assuming shapes having various lengths. In some useful methods in accordance with the present invention, the dimension DA of distal portion 120 of articulator 100 can be varied by urging relative motion between first wire 102 and second wire 104. Also in some useful methods in accordance with the present invention, a lateral stiffness of distal portion 120 of articulator 100 can be varied by urging relative motion between first wire 102 and second wire 104.

In the embodiment of FIG. 1, intermediate portion 124 of first wire 102 comprises a portion of wall 106 that defines a plurality of slots 132. A rib 134 of intermediate portion 124 of first wire 102 is defined by each adjacent pair of slots 132. A dimension DB of a most-distally located slot 136 is shown with dimension lines in FIG. 1. In the exemplary embodiment of FIG. 1, dimension DB is equal to about the outer diameter of first wire 102 minus the thickness of wall 106. A dimension DC of a most-proximally located slot 137 is also shown with dimension lines in FIG. 1. In the exemplary embodiment of FIG. 1, dimension DC is less than about one half the outer diameter of first wire 102.

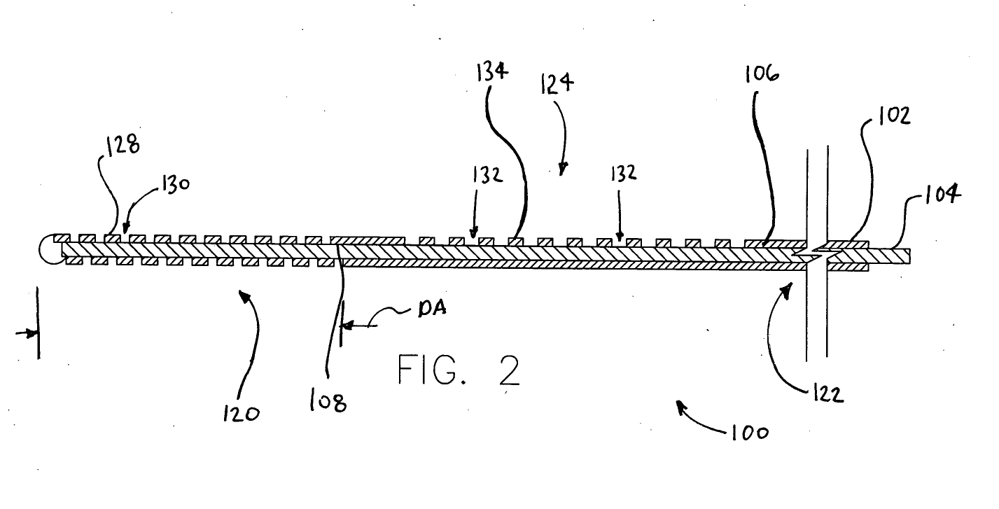

FIG. 2 is a cross sectional view of articulator 100 shown in the previous figure. In FIG. 2, a portion of second wire 104 is shown disposed within lumen 108 defined by wall 106 of first wire 102. In some useful embodiments of the present invention, intermediate portion 124 is adapted to assume one or more generally curved shapes. In the embodiment of FIG. 2, intermediate portion 124 of first wire 102 comprises a portion of wall 106 that defines a plurality of slots 132. A rib 134 of intermediate portion 124 of first wire is defined by each adjacent pair of slots 132. Slots 132 may be positioned and dimensioned such that intermediate portion 124 of first wire 102 can be urged to selectively assume various generally curved shapes.

With reference to FIG. 2, it will be appreciated that proximal portion 122 of first wire 102 comprises a solid portion of wall 106. Turns 128 of distal portion 120 of first wire 102 are also shown in FIG. 2. In the embodiment of FIG. 2, turns 128 are disposed with spaces 130 between adjacent turns 128. A dimension DA of distal portion 120 of articulator 100 is illustrated using dimension lines in FIG. 2.

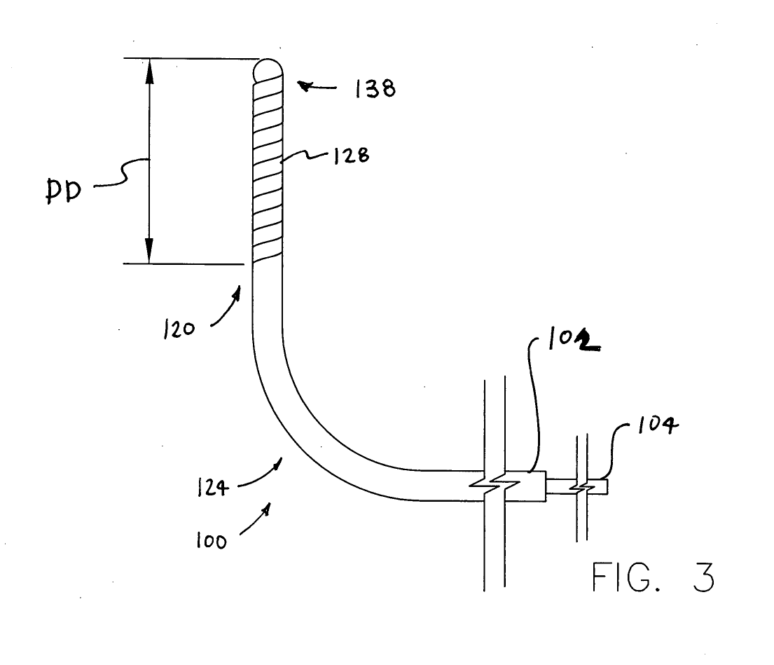

FIG. 3 is a side view of articulator 100 shown in the previous figure. In FIG. 3, intermediate portion 124 of first wire 102 is shown assuming a generally curved shape. In FIG. 3, turns 128 of distal portion 120 of second wire 104 are shown contacting one another. A dimension DD of distal portion 120 of articulator 100 is illustrated using dimension lines in FIG. 3. In the exemplary embodiment of FIG. 3, dimension DD is generally smaller than dimension DA shown in the previous figure.

In some useful embodiments of the present invention, articulator 100 is capable of assuming one or more shapes that will facilitate the performance of a selected surgical procedure. In some methods in accordance with this disclosure the step of urging intermediate portion 124 of articulator 100 to assume a curved shape aids a surgeon in aligning articulator tip 138 with the ostium of the coronary sinus of a human heart. Also, in some methods in accordance with the present disclosure, the step of urging distal portion 120 of articulator 100 to assume the second shape aids a surgeon in advancing articulator tip 138 into the ostium of a human heart once the articulator tip 138 is aligned with the ostium.

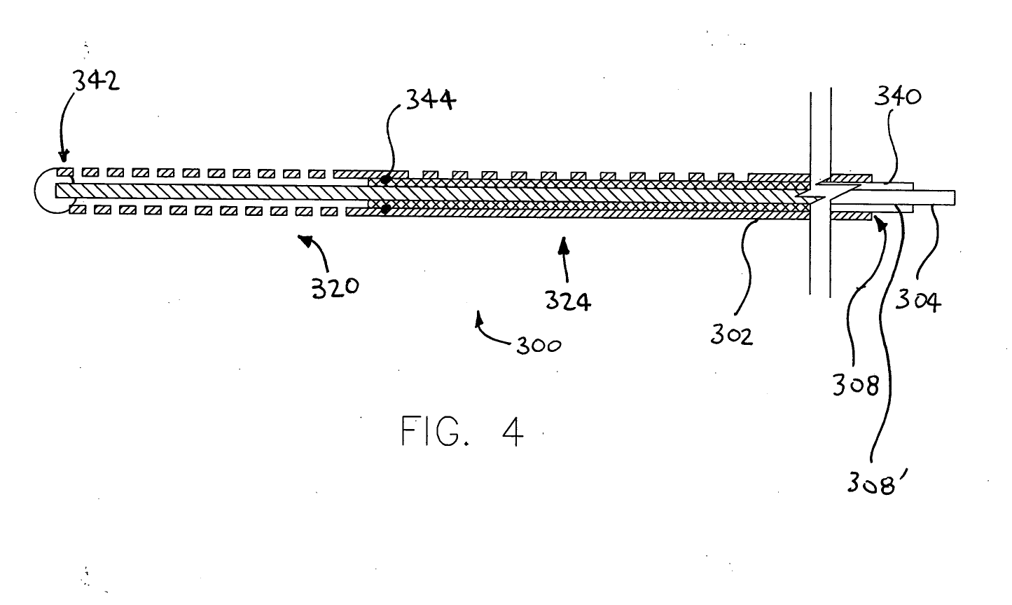

FIG. 4 is a side view of an articulator 300 comprising a first wire 302, a second wire 304 and a third wire 340. With reference to FIG. 4, it will be appreciated that a portion of third wire 340 is disposed within a first lumen 308 defined by first wire 302. Second wire 304 is disposed within a third lumen 308′ defined by third wire 340. A distal portion of second wire 304 is fixed to a distal portion of first wire 302 at a first joint 342. A distal portion of third wire 340 is fixed to first wire 302 at a second joint 344.

In some useful embodiments of the present invention, relative movement of the proximal end of second wire 304 relative to the proximal end of first wire 302 causes distal portion 320 of first wire 302 to contract in length. Also in some useful embodiments, relative movement between the proximal end of third wire 340 and the proximal end of first wire 302 causes intermediate portion 324 of first wire 302 to assume a generally bent shape.

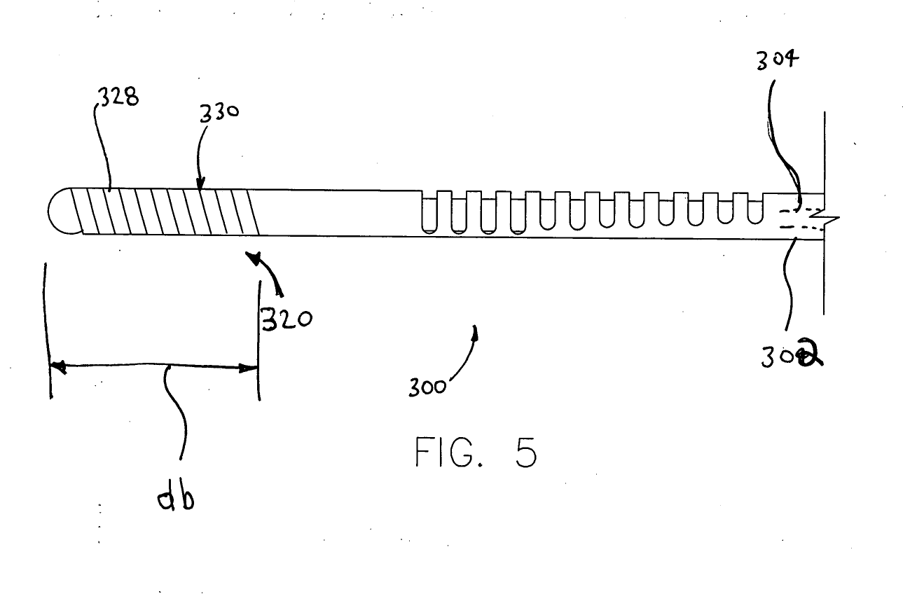

FIG. 5 is a side view of articulator 300 shown in the previous figure. In the embodiment of FIG. 5, the proximal end of second wire 304 has been urged proximally relative to the proximal end of first wire 302. With reference to FIG. 5, it will be appreciated that distal portion 320 of first wire 302 has a length db in the embodiment illustrated in FIG. 5. In the embodiment illustrated in FIG. 5, the spaces 330 between adjacent turns 328 of distal portion 320 of first wire 302 are generally smaller than those shown in the previous figure. In some embodiments of the present invention, the lateral stiffness of distal portion 320 of first wire 302 can be varied by varying the spacing between adjacent turns of distal portion 320.

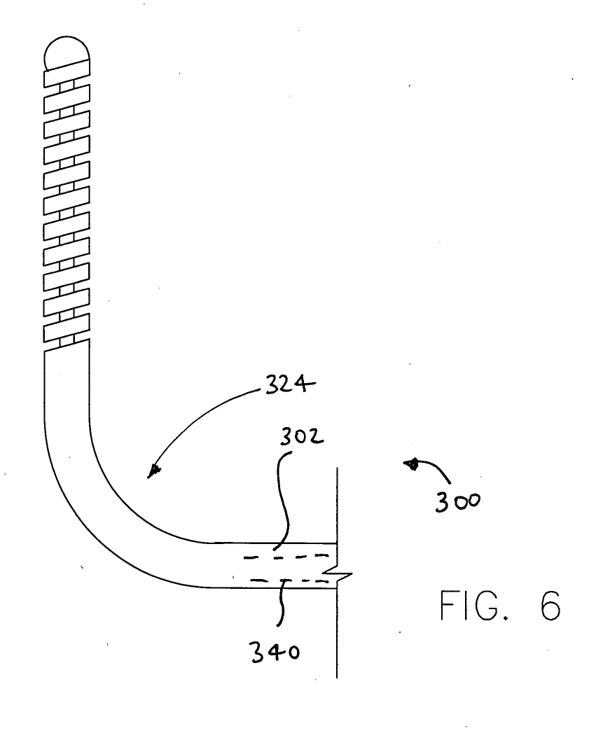

FIG. 6 is a side view of articulator 300 shown in the previous figure. With reference to FIG. 6, it will be appreciated that intermediate portion 324 of first wire 302 has assumed a generally curved shape. In some useful embodiments of the present invention, intermediate portion 324 of first wire 302 is capable of selectively assuming one or more generally curved shapes. In the embodiment of FIG. 6, intermediate portion 324 of first wire 302 can be caused to change shape by urging the proximal end of third wire 340 proximally relative to the proximal end of first wire 302.

An articulator in accordance with the present invention can be used to facilitate the performance of various medical procedures. For example, an articulator in accordance with the present invention may be used in conjunction with an intravascular catheter to perform percutaneous transluminal coronary angioplasty (PCTA). When this is the case, the articulator may be advanced through the patient's vasculature until its distal tip has moved past a restriction in a diseased vessel. In many cases, the articulator's path through the vascular system will be tortuous, requiring the articulator to change direction many times. Once the articulator is positioned so as to extend past the restriction, a balloon catheter may be threaded onto the articulator and urged distally until a balloon fixed near the distal end of the catheter is centered on the restriction in the diseased vessel. The balloon may then be inflated to open the restriction.

An articulator in accordance with the present invention may also be used in conjunction with the treatment of brain aneurysms. Approximately 25,000 patients suffer from ruptured intracranial aneurysms each year in North America. One treatment strategy is to treat the aneurysms before they rupture. Additionally, ruptured aneurysms may be treated to prevent rebleeding.

The endovascular treatment of brain aneurysms typically involves a two step approach. The first step is advancing the distal end of a catheter to the aneurysm site. The second step involves filling the aneurysm in some fashion or another. The aneurysm may be filled, for example, with a balloon. The balloon may be introduced into the aneurysm, inflated, detached, and left to occlude the aneurysm.

The aneurysm may also be filled with an embolism-forming device. The embolism forming device may comprise, for example, a soft, flexible coil having a small (e.g., 10 mil) outer diameter. The embolism forming device may also comprise absorbable tissue and/or fibrin. Within a short period of time after the filling of the aneurysm with the embolism forming device, a thrombus forms in the aneurysm and is shortly thereafter complemented with a collagenous material which significantly lessens the potential for aneurysm rupture.

An articulator in accordance with the present invention may also be use to facilitate endoscopic retrograde cholangio-pancreaticography (ERCP). ERCP procedures are often used when diagnosing and treating abnormal pathologies within the bile duct and the pancreatic duct. During such a procedure, an endoscope may be introduced into the mouth of the patient. The endoscope may be guided through the patient's alimentary tract or canal until an opening at the distal end of the endoscope is proximate the location for gaining access to the area to receive treatment. At this point, the endoscope allows for an articulator in accordance with the present invention, to access the targeted area. The distal end of the endoscope may be positioned proximate the papilla of vater leading to the bile duct and the pancreatic duct. The articulator is guided through a lumen defined by the endoscope until a distal tip of the articulator emerges from the opening at the distal end of the endoscope. The articulator may be inserted in an opening at a proximal end of the endoscope and guided through a lumen defined by the endoscope until it emerges from the distal end of the endoscope. The articulator the articulator may then be guided through the orifice to the papilla of vater (located between the sphincter of oddi) leading to the bile duct and the pancreatic duct. A catheter may be advanced over the articulator until the distal end of the catheter is positioned in a desired location. The catheter may be used to deliver fluoroscopic fluid to the bile duct and the pancreatic duct in order to diagnose pathological changes. The catheter may also be used to take biopsies, extract stones or insert stents to provide for an unobstructed bile or pancreatic flow. Once the articulator is properly positioned, the articulator helps to maintain the position of the catheter during these procedures.

Methods in accordance with the present invention are also possible with the step of urging the intermediate portion of the first wire to assume a curved shape aids a surgeon in aligning a tip of the articulator with the ostium of the coronary sinus of a human heart. Also, in some methods in accordance with the present disclosure, the step of urging the distal portion of the first wire to assume a shortened shape aids a surgeon in advancing articulator tip into the ostium of a human heart once the articulator tip is aligned with the ostium.

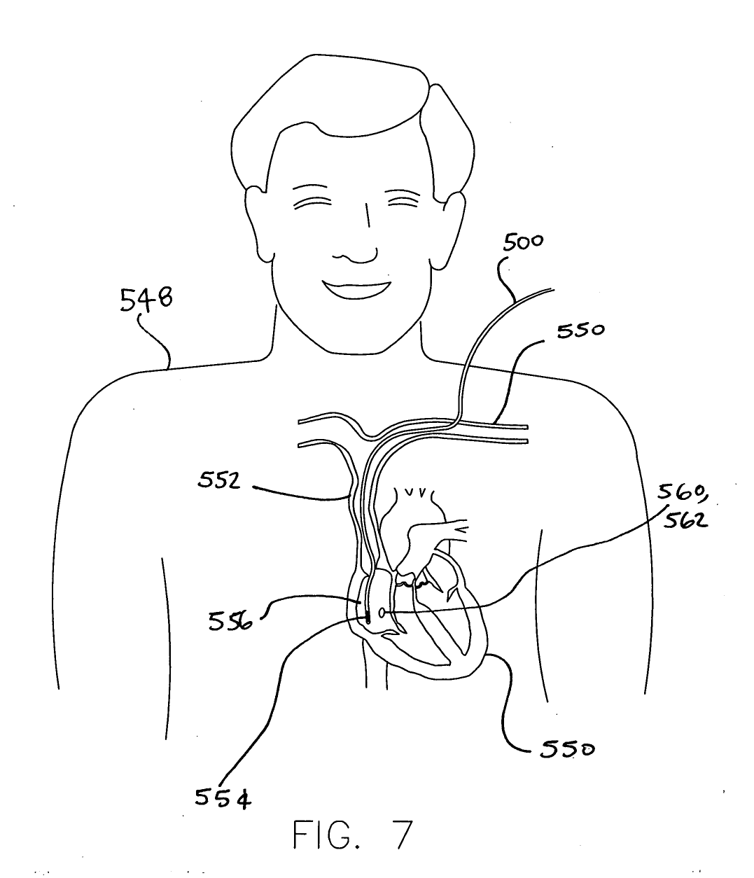

FIG. 7 is a ventral view of a patient 548. In FIG. 7, an articulator 500 is shown extending through the left subclavian vein 550 and the superior vena cava 552 so that a distal tip 554 of articulator 500 is disposed within the right atrium 556 of the heart 558. The ostium 560 of the coronary sinus 562 of heart 558 is also visible in FIG. 7.

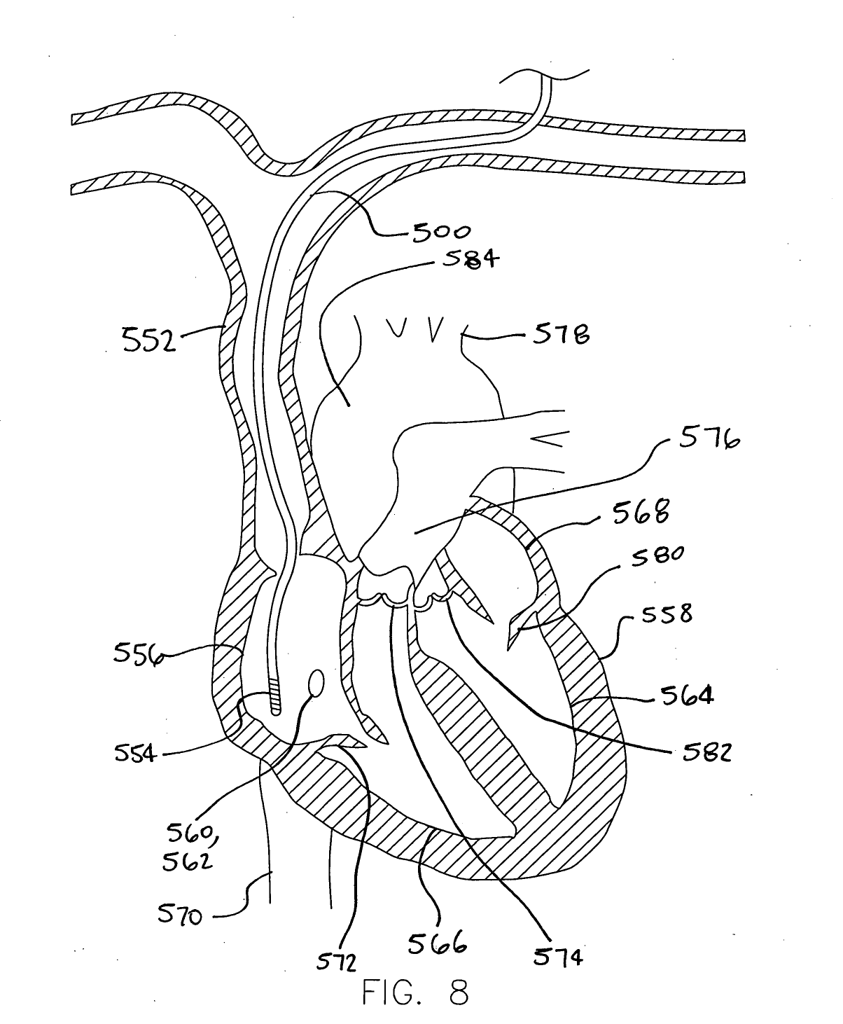

FIG. 8 is a partial cross sectional view of a heart 558. Heart 558 includes a left ventricle 564, a right ventricle 566, a left atrium 568, and a right atrium 556. In FIG. 8, an articulator 500 is shown extending through the superior vena cava 552 so that a distal tip 554 of articulator 500 is disposed within right atrium 556 of heart 558. The ostium 560 of the coronary sinus 562 of heart 558 is visible in FIG. 8. When heart 558 beats, blood from the upper portion of the body flows into right atrium 556 via the superior vena cava 552. Blood from the lower portion of the body flows into the right atrium 556 via the inferior vena cava 570. A tricuspid valve 572 is in fluid communication with both the right atrium 556 and the right ventricle 566. When tricuspid valve 572 opens, it allows blood to flow from right atrium 556 into right ventricle 566. During each heart beat, tricuspid valve 572 closes and right ventricle 566 contracts to pump blood through the pulmonary valve 574 into the pulmonary artery 576. The pulmonary artery carries blood to the lungs of the body.

After becoming oxygenated in the lungs, blood returns to the heart via a plurality of pulmonary veins 578 that are each in fluid communication with the left atrium 568. A mitrial valve 580 is in fluid communication with both left atrium 568 and left ventricle 564. Blood returning from the lungs via pulmonary veins 578 may pass through mitrial valve 580 into left ventricle 564. During each heart beat, mitrial valve 580 closes and left ventricle 564 contracts, pumping blood through an aortic valve 582 and into the aorta 584. After passing through the aorta 584, oxygenated blood is distributed throughout the body.

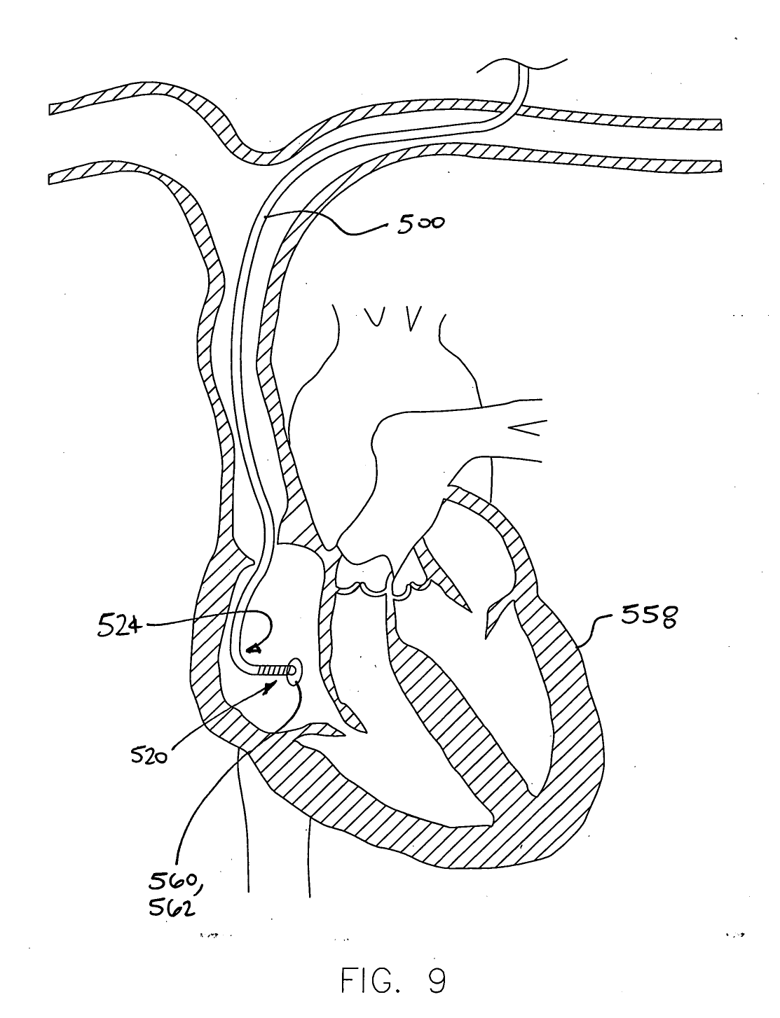

FIG. 9 is an additional partial cross sectional view of a heart 558 shown in the previous figure. In the embodiment of FIG. 9, intermediate portion 524 of articulator 500 has assumed a generally curved shape and distal portion 520 of articulator 500 is generally aligned with the ostium 560 of the coronary sinus 562 of heart 558. Some useful methods in accordance with the present invention may include the step of urging articulator 500 to assume one or more shapes that will facilitate the completion of a selected surgical procedure. For example, the step of urging intermediate portion 524 of articulator 500 to assume a curved shape may aid a surgeon in guiding articulator 500 into the coronary sinus.

In the embodiment of FIG. 9, distal portion 520 of articulator 500 has been urged to assume a generally shortened shape relative to the shape shown in the previous figure. In some useful embodiments of the present invention, the lateral stiffness of distal portion 520 may increase when distal portion 520 assumes a generally shorter shape. In some methods in accordance with the present disclosure, the step of advancing distal portion 520 into the ostium of a human heart may be facilitated by urging distal portion 520 of articulator 500 to assume a generally shortened shape.

Numerous characteristics and advantages of the invention covered by this document have been set forth in the foregoing description. It will be understood, however, that this disclosure is, in many respects, only illustrative. Changes may be made in details, particularly in matters of shape, size and ordering of steps without exceeding the scope of the invention. The invention's scope is, of course, defined in the language in which the appended claims are expressed.

What is claimed is:

1. An apparatus comprising:
a first wire, a second wire, and a third wire;
the third wire being disposed within a first lumen defined by the first wire;
the second wire being disposed in a third lumen defined the third wire;
a distal portion of the second wire being fixed to a distal portion of the second wire; and
a distal portion of the third wire being fixed to a distal portion of the first wire.

2. The apparatus of claim 1, wherein relative movement of a proximal portion of the second wire relative to a proximal portion of the first wire causes a distal portion of the first wire to contract in length.

3. The apparatus of claim 1, wherein relative movement between a proximal portion of the third wire and a proximal portion of the first wire causes an intermediate portion of the first wire to assume a curved shape.

4. The apparatus of claim 1, wherein the third wire is biased to assume a substantially straight shape.

5. The apparatus of claim 1, wherein the second wire is biased to assume a substantially straight shape.

6. The apparatus of claim 1, wherein:
the intermediate portion of the first wire is biased to assume a substantially straight shape; and
the intermediate portion of the first wire assumes a substantially curved shape when a proximal end of the third wire is urged proximately relative to a proximal end of the first wire.

7. An apparatus comprising:
a first wire, a second wire, and a third wire;
the third wire being disposed within a first lumen defined by the first wire;
the second wire being disposed in a third lumen defined the third wire; and
a distal portion of the apparatus being capable of assuming a first configuration and a second configuration;
the distal portion of the apparatus having a first lateral stiffness while the distal portion of the apparatus is assuming the first configuration;
the distal portion of the apparatus having a second lateral stiffness while the distal portion of the apparatus is assuming the second configuration;
the second radius of curvature being different from the first radius of curvature;
an intermediate portion of the apparatus being capable of assuming a first shape and a second shape;
the intermediate portion of the apparatus having a first radius of curvature while the intermediate portion of the apparatus is assuming the first shape;
the intermediate portion of the apparatus having a second radius of curvature while the intermediate portion of the apparatus is assuming the second shape;
the second radius of curvature being different from the first radius of curvature.

8. A method comprising the steps of:
providing an apparatus having a proximal portion, a distal portion, and an intermediate portion disposed between the proximal portion and the distal portion;
advancing the distal portion of the apparatus through vasculature of a body so that at least a distal tip of the apparatus is disposed proximate a target location within the body;
allowing the distal portion of the apparatus to assume a first configuration while the distal portion of the apparatus is being advanced through the vasculature of the body; and
urging the distal portion of the apparatus to assume a second configuration while the distal tip is disposed proximate the target location.

9. The method of claim 8, wherein:
the distal portion has a first lateral stiffness when the distal portion is assuming the first configuration;
the distal portion has a second lateral stiffness when the distal portion is assuming the second configuration; and
the second lateral stiffness is different from the first lateral stiffness.

10. The method of claim 8, wherein the second lateral stiffness is greater than the first lateral stiffness.

11. A method of claim 8, wherein:
the distal portion has a first length when the distal portion is assuming the first configuration;
the distal portion has a second length when the distal portion is assuming the second configuration; and
the second length is different from the first length.

12. The method of claim 8, wherein the second length is smaller than the first length.

13. The method of claim 8, further including the step of urging the intermediate portion of the apparatus to assume a curved shape.

14. The method of claim 8, wherein:
the intermediate portion has a first radius of curvature when the intermediate portion is assuming the first shape;
the intermediate portion has a second radius of curvature when the intermediate portion is assuming the second shape;
the second radius of curvature is greater than the first radius of curvature.

15. A method comprising the steps of:
providing an apparatus having a proximal portion, a intermediate portion, and an intermediate portion disposed between the proximal portion and the intermediate portion;
advancing the intermediate portion of the apparatus through vasculature of a body so that at least a intermediate tip of the apparatus is disposed proximate a target location within the body;
allowing the intermediate portion of the apparatus to assume a first shape while the intermediate portion of the apparatus is being advanced through the vasculature of the body; and
urging the intermediate portion of the apparatus to assume a second shape while the intermediate tip is disposed proximate the target location.

16. The method of claim 15, wherein:
the intermediate portion has a first radius of curvature when the intermediate portion is assuming the first shape;
the intermediate portion has a second radius of curvature when the intermediate portion is assuming the second shape;
the second radius of curvature is greater than the first radius of curvature.

17. A method comprising the steps of:
providing a stylet having a proximal portion, a distal portion, and an intermediate portion disposed between the proximal portion and the distal portion;
advancing the distal portion of the stylet through vasculature of a body so that at least a distal tip of the stylet is disposed within a chamber of a heart of the body;
allowing the distal portion of the stylet to assume a first shape while the distal portion of the stylet is being advanced through the vasculature of the body; and
urging the distal portion of the stylet to assume a second shape while the distal tip is disposed in the chamber of the heart.

18. The method of claim 17, wherein:
the distal portion has a first lateral stiffness when the distal portion is assuming the first shape;
the distal portion has a second lateral stiffness when the distal portion is assuming the second shape; and
the second lateral stiffness is different from the first lateral stiffness.

19. The method of claim 18, wherein the second lateral stiffness is greater than the first lateral stiffness.

20. A method of claim 17, wherein:
the distal portion has a first length when the distal portion is assuming the first shape;
the distal portion has a second length when the distal portion is assuming the second shape; and
the second length is different from the first length.

21. The method of claim 20, wherein the second length is smaller than the first length.

22. The method of claim 17, further including the step of urging the intermediate portion of the stylet to assume a curved shape while the distal tip is disposed in the chamber of the heart.

23. The method of claim 17, further including the step of urging the intermediate portion of the stylet to assume a curved shape comprises the step of urging the intermediate portion of the stylet to assume a radius of curvature that aligns the distal tip of the stylet with an astium of a great vein of the heart.

24. The method of claim 23, wherein:
the intermediate portion has a first radius of curvature when the intermediate portion is assuming the first shape;
the intermediate portion has a second radius of curvature when the intermediate portion is assuming the second shape;
the second radius of curvature is greater than the first radius of curvature.

25. The method of claim 17, wherein the chamber is the right atrium of the heart.

26. A stylet comprising:
a first wire, a second wire, and a third wire;
the third wire being disposed within a first lumen defined by the first wire;
the second wire being disposed in a third lumen defined the third wire; and
means for reconfiguring the distal and intermediate portions of the first wire from a first configuration for advancing within a vessel with minimum trauma to the vessel to a second configuration for entering the ostium of the cardiac sinus.

27. The sylet of claim 26, wherein the distal portion and the intermediate portion of the first wire fit within a right atrium of a human heart when the first wire assumes the second configuration.
